(19) United States
(12) Patent Application Publication
(10) Pub. No.: US 2013/0323332 A1
(43) Pub. Date: Dec. 5, 2013

(54) Hand and body moisturizing serum

(71) Applicant: Sherry May Raymond-Coblantz, Bend, OR (US)

(72) Inventor: Sherry May Raymond-Coblantz, Bend, OR (US)

(21) Appl. No.: 13/506,982

(22) Filed: May 31, 2012

(51) Int. Cl. A61K 8/97; A61Q 19/00

(52) U.S. Cl. 424/729

(57) ABSTRACT

An organic hand and body moisturizing serum comprised of; Sacha Inchi seed oil, Camellia seed oil, Sunflower seed oil, Jojoba oil, Palm stearic acid flakes, an emulsifying wax, Vitamin E d-alpha tocopherol, Lecithin, Rosemary oil, Tea tree oil, and Helichrysum oil or Evening Primrose seed oil. Another embodiment of the moisturizing serum would include Uviox made from hydrolyzed grape skin.

### CROSS-REFERENCE TO RELATED APPLICATIONS

None.

### STATEMENT REGARDING FEDERALLY SPONSORED RESEARCH OR DEVELOPMENT

None.

### BACKGROUND OF THE INVENTION

The present invention is a composition of oils and other natural products to strengthen and enhance the health of skin. The composition promotes a firmness to the skin and promotes dermal hydration for a stronger healthier skin.

There are many products and formulas for treating dry rough skin and just as many formulas to strengthen the skin. The prior art is replete with products and patents claiming skin protection and salvations for aging skin however the present invention combines a combination of organic ingredients for treating various skin disorders that is unique and effective in bringing about soft, moist skin and reducing wrinkles without harming the skin or the body.

Skin disorders, as the term is used herein, encompasses numerous skin conditions ranging in severity from severe dermatitis, severe dry skin, psoriasis, rosacea and other skin blemishes to less severe conditions, such as lack of adequate skin firmness, dermal hydration or scaling, which are nonetheless unsightly and may cause physical discomfort. There is a need for a skin treatment that enhances the health of the skin and rejuvenates the skin at the cellular level.

Until now, the treatment of skin disorders has been largely based on non-specific drugs, and only limited success has been achieved. Dermatitis, for example, which may be accompanied by severe scaling, fissures, edema, oozing, erosion, itching and thickening of the skin, commonly has been treated with corticosteroids. Such compounds provide symptomatic relief for some patients. Steroids, however, are known to produce many local and systemic side effects, and their long term use may not be desirable.

Similarly Vitamin D is therapeutically effective in treating certain skin disorders, but only in dosages which are associated with undesirable side effects. Vitamin D at the dose ranges used in currently marketed topical preparations is not therapeutically effective against contact dermatitis. Other formulations for treating skin disorders have either been ineffective or have caused significant irritation to the skin. Several formulations have been proposed to overcome the disadvantages of the prior art, both for treating skin disorders, and for use in cosmetics in order to prevent skin irritation and clear blemishes.

There are numerous patents issued for the care and condition of skin. Each patent uses a variety of ingredients with the combination providing some form of skin treatment. The list is exhaustive, and includes such patents as: U.S. Pat. No. 7,306,810 issued Dec. 11, 2007 to Spencer discloses a skin cream which comprises at least one anti-oxidant, an anti-inflammatory agent, an exfoliant, and an agent to protect against UV irradiation and the cream is made from either oils or creams to promote adsorption of the active ingredients and to increase vibrancy of the skin tone. U.S. Pat. No. 6,281,236 issued Aug. 28, 2001 to Farber discloses a skin cream composition containing allantoin and an emulsifier with improved stability coming from the adjustment of the pH to a range of 4.5 to 5.8. The lower pH preserves the stability of the allantoin and the functionality of the emulsifier system is maintained. U.S. Pat. No. 6,572,868, issued Jun. 3, 2003 to Sandra E. Cope, discloses a restructuring complex for cosmetic compositions. The composition comprises safe and effective amounts of carrageenans, borage seed oil, squalane, ceramide 3, ceramide 6; red algae extract, dipalmitoyl hydroxproline, and oleuropein. U.S. Pat. No. 6,193,987, issued Feb. 27, 2001 to M. H. Harbeck, discloses a lubricating composition for the hands and skin. The composition has as its constituents a mixture of organic safflower oil, flaxseed oil, tincture of benzoin, and organic beeswax. And, U.S. Pat. No. 6,479,043, issued Nov. 12, 2002 to Tietjen et al., discloses a depilatory composition. The composition includes emollients, skin conditioners, buffering agents, viscosity increasing agents, emulsion stabilizers, pH adjusters, chelating agents, fragrance, color, lubricants, propellants, or biological agents.

Various topical formulations and oral regimens of vitamins and herbs have been proposed for the treatment of skin conditions. U.S. Pat. No. 6,228,387, issued May 8, 2001 to M. Borod, describes a first composition for topical application and a second composition for oral administration for the treatment of hemorrhoids. The topical composition includes several herbs and vitamins, including grape seed extract and vitamin E, and in one embodiment, a few drops of Essential Oil of Chamomile. Vitamin E occurs naturally as a mixture of tocopherols, the most active being a-tocopherol, used externally, vitamin E is healing to the skin, being used for protection from sun damage, reducing facial lines and wrinkles, and improving skin smoothness, being used as an additive to massage oils and face creams.

In the prior art, there is little or no distinction between the production of the various compound used in the numerous skin treatments. The present invention provides for a combination of organic ingredients blended from various oils, extracts and waxes to provide for a unique and exceptional skin treatment to enhance the health of the skin that can be used everyday with no adverse side effects. The use of organic products, including wild field products eliminates additives such as synthetic pesticides and chemical fertilizers. The products used in the blended compound do not contain genetically modified organisms, and are not processed using irradiation, industrial solvents, or chemical food additives. By eliminating products that are made or produced with pesticides, genetically altered organisms or chemical fertilizer the effects of the product are enhanced and this limits any potential harmful side effects to the skin.

### BRIEF SUMMARY OF THE INVENTION

A hand and body serum, comprising approximately: 28%-43% Sacha Inchi seed oil, 15%-20% Camellia seed oil, 10%-20% sunflower seed oil, 10%-18% Jojoba oil, 5%-%15 Palm stearic acid flakes, 2%-8% emulsifying wax made from either palm kernel or coconut (cetylstearyl alcohol), 2%-6% Palm derived Vitamin E d-alpha tocopherol, 2%, 6% Lecithin, 1%-4% Rosemary oil, 1%-4% Tea tree oil, and 1%-4% Helichrysum oil or Evening Primrose seed oil.

Another embodiment of the hand and body moisturizer, would include the above component with the addition of approximately 2%-10% Uviox made from hydrolyzed grape skin.

The above percentages are volume measurements of the various components added making up the moisturizing serum.

### BRIEF DESCRIPTION OF THE SEVERAL VIEWS OF THE DRAWING

None

### DETAILED DESCRIPTION OF THE INVENTION

Various embodiments of the invention will now be described. The following descriptions provide specific details for a thorough understanding and enabling description of these embodiments. Additionally, some well-known structures or functions may not be shown or described in detail, so as to avoid unnecessarily obscuring the relevant description of the various aspects and embodiments of the invention.

The terminology used in the description presented below is intended to be interpreted in its broadest reasonable manner, even though it is being used in conjunction with a detailed description of certain specific embodiments of the invention. Certain terms may even be emphasized herein; however, any terminology intended to be interpreted in any restricted manner will be overly and specifically defined as such in this Detailed Description section.

The present invention is a topical blend of oil, extracts and waxes created by combining several organic or natural ingredients, all of which are plant based. By combining the various ingredients the applicant has found significant improvement in a user's skin. The skin is more youthful, firmer, and improves the health of the skin cells. The present invention produces a firmer tighter healthier skin.

The moisturizing serums are produced using organic products, which means: the ingredients are grown and processed organically. For example, organic farming practices are designed to encourage soil and water conservation and reduce pollution. Farmers who grow organic produce don't use conventional methods to fertilize or control weeds. For example, rather than using chemical weed killers, organic farmers may conduct more sophisticated crop rotations and spread mulch or manure to keep weeds at bay. Additionally, the ingredients are organically processed which includes; cold press extraction or other means for processing the various plants to produces the oils without using or adding chemicals in the extraction process. The following serums were developed using organic or natural ingredients and have produced exceptional results. The claims are not limited to organic ingredients but the applicant cannot confirm the same positive results using non-organic products.

In the first embodiment, a hand and body moisturizing serum, comprising approximately: 28%-43% Sacha Inchi seed oil, 15%-20% Camellia seed oil, 10%-20% sunflower seed oil, 10%-18% Jojoba oil, 5%-%15 Stearic Acid Flakes, 2%-8%, an emulsifying wax derived from either palm kernel or coconut oil (cetylstearyl alcohol), 2%-6% Palm derived Vitamin E d-alpha Tocopherol, 2%-6% Lecithin, 1%-4% Rosemary oil, 1%-4% Tea tree oil, and 1%-4% Helichrysum oil or Evening Primrose seed oil. All of the ingredients of this skin product are organically or naturally produced and processed and come from many countries.

Each ingredient is listed by volume. Since the specific weights and densities of the various oils are similar, volume measurements closely represent percentages by weight. The d-alpha tocopherol, the Rosemary extract, and Tea Tree oil contributes to the stability of the blended oils and are high in antioxidants. The tocopherol is known to protect the skin from UV rays and reactive radicals. The tocopherol also provides some moisturizing benefits to the skin.

Sacha Inchi seed oil is produced from the Sacha Inchi plant and the oil from the seeds are high in fatty acids and antioxidants. The variety of fatty acids that are found in the seed oil, including; Linoleic and Alfa Linoleic, Oleic, Palmitic, and Stearic acids. In addition to being used as a conditioning oil it is also beneficial in the treatment of burns, wounds, lesions, and sun damaged skin.

Camellia seed oil is a highly penetrating and virtually odorless oil that possesses a slight herbal aroma and offers unique UV protection. It is virtually clear with a slight yellow hue. Camellia Oil is light in texture and absorbs quickly into the skin. Approximately 85% of the fatty acid composition contained in Camellia Oil is in the form of monounsaturated oleic acid. Camellia Oil is also a rich source of other omega 3,6 and 9 fatty acids plus Vitamin E and polyphenols. The Camellia seed oil has been shown to have the following properties; penetrates quickly, moisturizes, emits a light herbal aroma, is rich in fatty and essential fatty acids, contains tocopherol a form of Vitamin E, and contains an abundance of polyphenols.

Jojoba oil is a mixture of long chain monounsaturated liquid wax esters. Jojoba oil is extracted from seeds of the jojoba plant, scientifically known as Simmondsia chinensis, which are indigenous to southern United States and northern Mexico. Jojoba oil has a golden color whereas processed jojoba oil is clear and transparent. It has a relatively long shelf life as it is a relatively stable liquid. It does not oxidize easily and will not turn rancid compared to other oils.

Rosemary essential oil improves circulation and has a rejuvenating effect on the skin. Rosemary is a common ingredient used in many cosmetics, including skin toners, creams, soaps and hair products. The essential oil is known to assist in the prevention of age-related skin damage, such as wrinkles. The oil is derived from the flowering top and leaves of the plant. Organic extraction requires steam distillation of the oil from the plant.

Helichrysum italicum oil, also known as Immortelle oil, which has properties that are beneficial to the skin, including; acting as an anti-inflammatory and soothes burns and raw chapped skin. It is used as a stabilizing and preserving agent in the present compound as well as a contributing to the benefits to the skin. The serum can be made by replacing the Helichrysum italicum oil with Evening Primrose oil. Evening Primrose oil has beneficial qualities for improving the skin and protecting the skin from ultraviolet (UV) light.

The above blended oils are then applied topically to the skin. The blended oils can be applied every day or as needed. The moisturizing serum is best applied after a show or after the user washes their skin. The skin should be free of soap, makeup or other skin products. The compound should be allowed to time to be absorbed by the skin and will depend upon the skin type but approximately 20 minutes. After absorption, other skin products, such as sun screen, can be applied.

Another embodiment of the hand and body moisturizing serum, would include the above component with the addition of approximately 2%-10% Uviox made from hydrolyzed grape skin.

I claim:

1. A hand and body moisturizing serum comprising:
28%-43% Sacha Inchi seed oil;
15%-20% Camellia seed oil;
10%-20% sunflower seed oil;
10%-18% Jojoba oil;
5%-%15 Stearic Acid Flakes;
2%-8% emulsifying wax made from either palm kernel or coconut (cetylstearyl alcohol);
2%-6% Vitamin E d-alpha Tocopherol;
2%-6% Lecithin;
1%-4% Rosemary oil;
1%-4% Tea tree oil; and
1%-4% Helichrysum oil.

2. The hand and body moisturizing serum of claim 1, wherein 1%-4% Evening Primrose oil is used in place of the Helichrysum oil.

3. A hand and body moisturizing serum comprising:
28%-43% Sacha Inchi seed oil;
15%-20% Camellia seed oil;
10%-20% sunflower seed oil;
10%-18% Jojoba oil;
5%-%15 Stearic Acid Flakes;
2%-10% Uvoix made from hydrolyzed grape skin;
2%-8% emulsifying wax made from either palm kernel or coconut (cetylstearyl alcohol);
2%-6% Vitamin E d-alpha Tocopherol;
2%-6% Lecithin;
1%-4% Rosemary oil;
1%-4% Tea tree oil; and
1%-4% Helichrysum oil or Evening Primrose seed oil.

4. The hand and body moisturizing serum of claim 3 wherein 1%-4% Evening Primrose oil is used in place of the Helichrysum oil.
